(19) United States
(12) Patent Application Publication
(10) Pub. No.: US 2007/0161792 A1
(43) Pub. Date: Jul. 12, 2007

(54) Methods for preparing 2,3,5,6-substituted 3h-pyrimidin-4-ones

(75) Inventors: Irina Shcherbakova, Midvale, UT (US); Manuel F. Balandria, Sandy, UT (US); Guangfei Huang, Alchua, FL (US); Otto Geoffroy, Alchua, FL (US); John Fox, Salt Lake City, UT (US); Satheesh K. Nair, Missouls, MT (US)

Correspondence Address: Matthew S Bethards;Stoel Rives, One Utah Center, 201 South Main Street Suite 1100, Salt Lake City, UT, 84111, US

(73) Assignee: NPS PHARMACUETICALS, INC, SALT LAKE CITY, UT (US)

(21) Appl. No.: 10/551,920

(22) PCT Filed: Apr. 7, 2004
(86) PCT No.: PCT/US04/10639

§ 371 Date: Nov. 20, 2006

Related U.S. Application Data
(60) Provisional application No. 60/460,859, filed on Apr. 7, 2003.
(60) Provisional application No. 60/479,323, filed on Jun. 18, 2003.

(51) Int. Cl. C07D 239/88; C07D 239/36

(52) U.S. Cl. 544/319; 544/287

(57) ABSTRACT

Pyrimidinone compounds are disclosed. Methods of preparing the pyrimidinone compounds are also disclosed.

### TECHNICAL FIELD

The present invention relates to methods for preparing of 2,3,5,6-substituted 3H-pyrimidin-4-ones.

### BACKGROUND OF THE INVENTION

Polysubstituted 3H-pyrimidin-4-ones are pharmacophores present in many biologically active compounds [see Salimbeni et al., J. Med. Chem., 1995, 38, 4806-4820; Jezewski et al., J. Heferocycic Chem. 2001, 38, 645-648; Madhavan et al., Biorg. Med. Chem. 2002, 10, 2671-2680].

### DETAILED DESCRIPTION OF PREFERRED EMBODIMENTS

Methods are disclosed for preparing of 2,3,5,6-substituted 3H-pyrimidin-4-ones. Embodiments of 2,3,5,6-substituted 3H-pyrimidin-4-ones are represented by Structure I. Structure I has the following chemical formula: 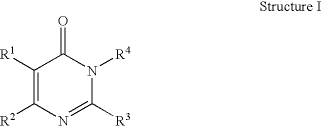
wherein:
R¹ and R² are independently one of: H, halogen, CN, CF₃, lower alkyl, cycloalk, and aryl; or R¹ and R² are together —(CH₂)ₙ— and n is 5, 4, or 3;
R³ is an aryl group, which may have 1 to 4 substituents in the aryl ring and each substituent is one of: H, halogen, CN, CF₃, OCF₃, lower alkyl, N(lower alkyl)₂, lower alkoxy, OH, OC(O)-lower alkyl, OC(O)-lower alkylamino, and OC(O)-lower alkyl-N(lower alkyl)₂;
R⁴ is one of H, lower alkyl, or a group of the formula —(CH₂)ₙ—R⁵ wherein n is 0, 1, or 2, R⁵ is an aryl group which may have 1 to 3 substituents on the aryl ring and each substituent is one of: H, halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, and OC(O)-lower alkyl-N(lower alk)₂; and
   pharmaceutically acceptable salts and complexes thereof.

In embodiments wherein R¹ and R² are independently selected, R¹ and R² may be one of: lower alkyl, cycloalkyl and aryl or one of lower alkyl and cycloalkyl. In embodiments wherein R¹ and R² are together —(CH₂)ₙ—, n may be 4 or 3.

In embodiments wherein R³ is a phenyl group, the phenyl ring may have 1 to 3 substituents which are one of: H, halogen, lower alkyl, lower alkoxy and OH. Also, in other embodiments wherein R³ is a phenyl group, the phenyl ring may have 1 to 3 substituents which are one of: H, halogen and OH.

In embodiments wherein R⁴ is a group of the formula —(CH₂)ₙ—R⁵, n is 1 or 2, and R⁵ is an aryl group, 1 to 3 substituents on the aryl ring are one of: H, halogen, lower alkyl or lower alkoxy. Also, in embodiments wherein R⁴ is a group of the formula —(CH₂)ₙ—R⁵, n is 2, and R⁵ is an aryl group, 1 to 3 substituents on the aryl ring are one of: H, halogen, lower alkyl and lower alkoxy.

“Alk” refers to either alkyl or alkenyl. “Lower alk” refers to either lower alkyl or lower alkenyl, preferably lower alkyl.

“Alkenyl” refers to an optionally substituted hydrocarbon group containing at least one carbon-carbon double bond between the carbon atoms and containing 2-6 carbon atoms joined together. The alkenyl hydrocarbon group may be straight-chain. Straight-chain alkenyl preferably has 2 to 4 carbons.

“Alkyl” refers to an optionally substituted hydrocarbon group joined by single carbon-carbon bonds and having 1 to 6 carbon atoms joined together. The alkyl hydrocarbon group may be straight-chain or contain one or more branches. In some embodiments, branched- and straight-chain alkyl groups have 1 to 4 carbons, each of which may be optionally substituted. Alkyl substituents are independently one of: lower alkyl, unsubstituted aryl, OH, NH₂, NH-lower alkyl, and N(lower alkyl)₂. In some embodiments, no more than two substituents are present. For example, alkyl may be a lower alkyl which is unsubstituted branched- or straight-chain alkyl having 1 to 4 carbons.

“Cycloalk” refers to an optionally substituted cyclic alkyl or an optionally substituted non-aromatic cyclic alkenyl and includes monocyclic and multiple ring structures such as bicyclic and tricyclic. The cycloalkyl has 3 to 15 carbon atoms. In one embodiment, cycloalkyl has 3 to 5 carbon atoms. Optional substituents for cycloalk are independently selected from the group described above for alkenyl. In one embodiment, no more than three substituents are present. In another embodiment, the cycloalk is unsubstituted. For example, the cylcoalk may be unsubstituted cyclic alkyl. Examples of suitable cycloalkyl groups include cyclopropyl and cyclobutyl.

“Aryl” refers to an optionally substituted aromatic group with at least one ring having a conjugated or fused ring system. Aryl includes carbocyclic aryl, heterocyclic aryl and biaryl groups, all of which may be optionally substituted. The aryl may be either optionally substituted phenyl or optionally substituted pyridyl.

“Alkoxy” refers to oxygen joined to an unsubstituted alkyl 1 to 4 carbon atoms in length. In one embodiment, the oxygen is joined to an unsubstituted alklyl 1 to 2 carbons in length. For example, the alkoxy may be methoxy.

The compounds of Structure I wherein R¹ is hydrogen can be prepared using standard techniques [for example, see Eason et al., J. Chem Soc. 2991-3000 (1931); Gardner et al., J. Org. Chem. 59, 6245-6250 (1994), Tice et al., Tetrahedron, 57, 2689-2700 (2001)]. The compounds of Structure I of the present invention wherein R¹, R² and R⁴ are substituents other than hydrogen, and R³ is an aryl group which may have substituents in the aryl ring, can be prepared by Scheme I involving a method of cyclizing an appropriate acetic acid 2-(1-alkyl-2-R⁴-carbamoyl-alk-1-enylcarbamoyl)-phenyl ester. A chemical synthesis for such compounds by Scheme I is a novel approach to 2,3,5,6-substituted 3H-pyrimidin-4-ones which is an improvement in the art.

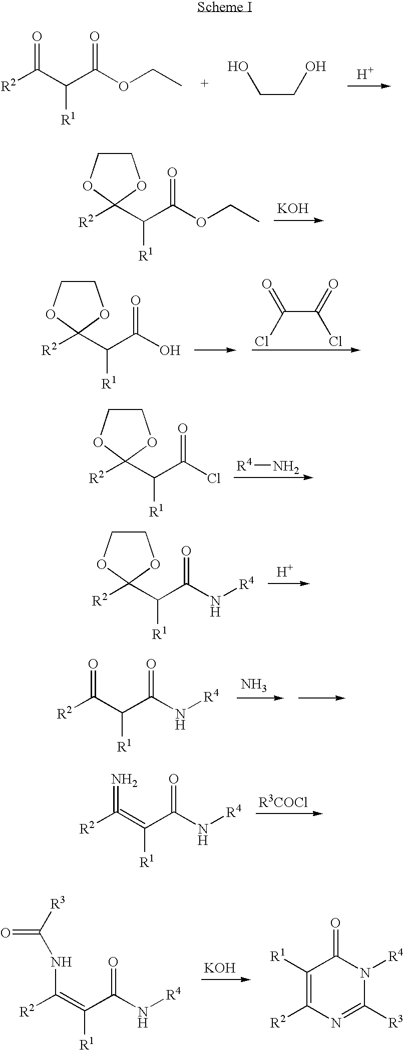

The chemical synthesis involves a method of making acetic acid 2-(1-alkyl-2-R⁴-carbamoyl-alk-1-enylcarbamoyl)-phenyl esters of Structure II by standard techniques which includes acylation of an appropriate 3-amino-2-alkyl-alk-2-enoic acid R⁴-amide of Structure III. 3-Amino-2-alkyl-alk-2-enoic acid R⁴-amide of Structure III can be prepared by reacting 2-alkyl-3-oxo-R⁴-amide with anhydrous ammonia on catalysis by anhydrous aluminum chloride. 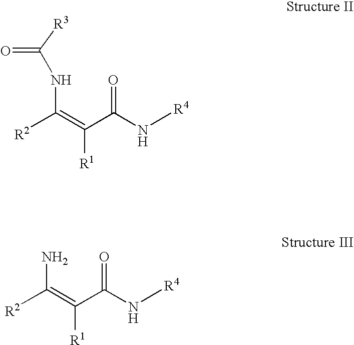
wherein:
R¹ and R² are independently one of: lower alkyl, and cycloalk; or R¹ and R² are together —(CH₂)ₙ— and n is 5, 4, or 3;
R³ is an aryl group, which may have 1 to 4 substituents in the aryl ring and each substituent is one of: H, halogen, lower alkyl, NH(lower alkyl), lower alkoxy, OH, OC(O)-lower alkyl, OC(O)-lower alkylamino, and OC(O)-lower alkyl-N(lower alkyl)₂;
R⁴ is one of H, lower alkyl, and a group of the formula —(CH₂)ₙ—R⁵ wherein n is 0, 1, or 2, and wherein R⁵ is an aryl group which may have 1 to 3 substituents on the aryl ring and each substituent is one of: H, halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, and OC(O)-lower alkyl-N(lower alk)₂.

In embodiments wherein R¹ and R² are independently selected, R¹ and R² may be one of: lower alkyl and cycloalkyl. In embodiments wherein R¹ and R² are together —(CH₂)ₙ—, n may be 4 or 3.

In embodiments wherein R³ is a phenyl group, the phenyl ring may have 1 to 3 substituents which are one of: H, halogen, lower alkyl, lower alkoxy and OH. Also, in other embodiments wherein R³ is a phenyl group, the phenyl ring may have 1 to 3 substituents which are one of: H, halogen and OH.

In embodiments wherein R⁴ is a group of the formula —(CH₂)ₙ—R⁵, n is 1 or 2, and R⁵ is an aryl group, 1 to 3 substituents on the aryl ring are one of: H, halogen, lower alkyl or lower alkoxy. Also, in embodiments wherein R⁴ is a group of the formula —(CH₂)ₙ—R⁵, n is 2, and R⁵ is an aryl group, 1 to 3 substituents on the aryl ring are one of: H, halogen, lower alkyl and lower alkoxy.

The following is a detailed description of the steps shown in Scheme I for synthesizing 2,3,5,6-Substituted 3H-Pyrimidin-4-ones. The first step involves reacting 2-alkyl-3-oxo-alkylic acid alkyl ester with ethylene glycol and p-toluenesulfonic acid monohydrate to obtain 2-(2-alkyl-[1,3]dioxolan-2-yl)-alkanoic acid alkyl ester. This step is followed by hydrolysis of 2-(2-alkyl-[1,3]dioxolan-2-yl)-alkanoic acid alkyl ester. Next, 2-(2-alkyl-[1,3]dioxolan-2-yl)-alkanoic acid is reacted with oxalyl chloride followed by reaction with a primary amine to obtain 2-(2-alkyl-[1,3]dioxolan-2-yl)-N-R⁴-alkanamide. Then, 2-(2-alkyl-[1,3]dioxolan-2-yl)-N-R⁴-alkanamide is reacted with p-toluenesulfonic acid monohydrate to obtain 2-alkyl-3-oxo-R⁴-amide. The next step involves reacting 2-alkyl-3-oxo-R⁴-amide with anhydrous ammonia on catalysis by anhydrous aluminum chloride to obtain an appropriate 3-amino-2-alkyl-alk-2-enoic acid R⁴-amide. Acylation of the appropriate 3-amino-2-alkyl-alk-2-enoic acid R⁴-amide is then performed to obtain an appropriate acetic acid 2-(1-alkyl-2-R⁴-carbamoyl-alk-1-enylcarbamoyl)-phenyl ester. Finally, acetic acid 2-(1-alkyl-2-R⁴-carbamoyl-alk-1-enylcarbamoyl)-phenyl ester, which is an example of an appropriate carbamide, is then cyclized.

Scheme II, below, provides another method for synthesizing 2,3,5,6-substituted 3H-pyrimidin-4-ones. More particularly, the compounds of Structure I of the present invention wherein R¹ is an alkyl group which contains more than one branch, R² is methyl, R⁴ is a substituent other than hydrogen, and R³ is an aryl group which may-have substituents in the aryl ring, can be prepared by Scheme II provided below. Scheme II involves a method of reacting an appropriate 3-aroylamino-2-alkyl-but-3-enoic acid methyl ester (another example of an appropriate carbamide) with a primary amine and the Grignard reagent.

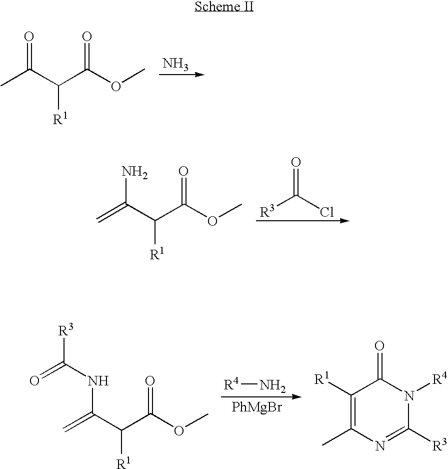

The chemical synthesis involves a method of making 3-R³-carbamoylamino-2-alkyl-but-3-enoic acid methyl ester of Structure IV by standard techniques which includes acylation of an appropriate 3-amino-2-alkyl-but-3-enoic acid methyl ester of Structure V. 3-Amino-2-alkyl-but-3-enoic acid methyl ester of Structure V can be prepared by reacting 2-alkyl-3-oxo-butyric acid methyl ester with liquid ammonia. 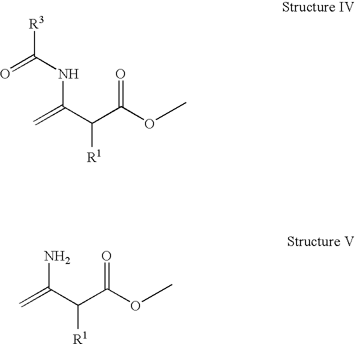
wherein:
R¹ is one of: lower alkyl and cycloalk;
R³ is aryl group, which may have 1 to 4 substituents in the aryl ring and each substituent is one of: H, halogen, lower alkyl, NH(lower alkyl), lower alkoxy, OH, OC(O)-lower alkyl, OC(O)-lower alkylamino, and OC(O)-lower alkyl-N(lower alkyl)₂; and
R⁴ is one of H, lower alkyl, and a group of the formula —(CH₂)ₙ—R⁵ wherein n is 0, 1, or 2, and wherein R⁵ is an aryl group which may have 1 to 3 substituents and each substituent is one of: H, halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, and OC(O)-lower alkyl-N(lower alk)₂.

In some embodiments of the compounds having the structure shown in Structure IV and V, R³ is a phenyl group which may have 1 to 3 substituents on the phenyl ring and each substituent is one of: H, halogen, lower alkyl, lower alkoxy and OH. In other embodiments, R³ is a phenyl group which may have 1 to 3 substituents on the phenyl ring and each substituent is one of: H, halogen and OH.

In some embodiments of compounds having the structure shown in Structure IV and V, R⁴ is a group of the formula —(CH₂)ₙ—R⁵ wherein n is 1 or 2, and wherein R⁵ is an aryl group which may have 1 to 3 substituents on the aryl ring and each substituent is one of: H, halogen, lower alkyl and lower alkoxy. In other embodiments, R⁴ is a group of the formula —(CH₂)ₙ—R⁵ wherein n is 2, and wherein R⁵ is an aryl group which may have 1 to 3 substituents on the aryl ring and each substituent is one of: H, halogen, lower alkyl and lower alkoxy.

In addition to an appropriate 3-aroylamino-2-alkyl-but-3-enoic acid methyl ester or more generically, 3-R³-carbamoylamino-2-alkyl-but-3-enoic acid methyl ester, another example of an appropriate carbamide for cyclizing to form 2,3,5,6-substituted 3H-pyrimidin-4-ones is 2-isopropyl-3-(2-methoxy-benzoylamino)-but-3-enoic acid methyl ester or more generically 2-alkyl-3-(2-alkoxy-benzoylamino)-but-3-enoic acid methyl ester as detailed below in Example 18.

Examples of 2,3,5,6-Substituted 3H-Pyrimidin-4-ones include:
2-(2-hydroxy-phenyl)-5,6-dimethyl-3-phenethyl-3H-pyrimidin-4-one;
3-[2-(2-fluoro-phenyl)-ethyl]-2-(2-hydroxy-phenyl)-5,6-dimethyl-3H-pyrimidin-4-one;
3-[2-(3-fluoro-phenyl)-ethyl]-2-(2-hydroxy-phenyl)-5,6-dimethyl-3H-pyrimidin-4-one;
3-[2-(4-fluoro-phenyl)-ethyl]-2-(2-hydroxy-phenyl)-5,6-dimethyl-3H-pyrimidin-4-one;
5-ethyl-2-(2-hydroxy-phenyl)-6-methyl-3-phenethyl-3H-pyrimidin-4-one;
5-ethyl-3-[2-(2-fluoro-phenyl)-ethyl]-2-(2-hydroxy-phenyl)-6-methyl-3H-pyrimidin-4-one;
5-ethyl-3-[2-(3-fluoro-phenyl)-ethyl]-2-(2-hydroxy-phenyl)-6-methyl-3H-pyrimidin-4-one;
5-ethyl-3-[2-(4-fluoro-phenyl)-ethyl]-2-(2-hydroxy-phenyl)-6-methyl-3H-pyrimidin-4-one;
3-[2-(3-fluoro-phenyl)-ethyl]-2-(2-hydroxy-phenyl)-6-methyl-5-propyl-3H-pyrimidin-4-one;
3-[2-(3-fluoro-phenyl)-ethyl]-2-(2-hydroxy-phenyl)-5-isopropyl-6-methyl-3H-pyrimidin-4-one;
3-[2-(3-fluoro-phenyl)-ethyl]-2-(2-methoxy-phenyl)-5-isopropyl-6-methyl-3H-pyrimidin-4-one;
3-[2-(2-fluoro-phenyl)-ethyl]-2-(2-hydroxy-phenyl)-5-isopropyl-6-methyl-3H-pyrimidin-4-one;
2-(2-hydroxy-phenyl)-5-methyl-3-phenethyl-6-trifluoromethyl-3H-pyrimidin-4-one;
2-(2-hydroxy-phenyl)-3-phenethyl-5,6,7,8-tetrahydro-3H-quinazolin-4-one;
3-[2-(3-fluoro-phenyl)-ethyl]-2-(2-hydroxy-phenyl)-5,6,7,8-tetrahydro-3H-quinazolin-4-one;
5-cyclopropyl-3-[2-(3-fluoro-phenyl)-ethyl]-2-(2-hydroxy-phenyl)-6-methyl-3H-pyrimidin-4-one;
2-(2-hydroxy-phenyl)-3-phenethyl-3,5,6,7-tetrahydro-cyclopentapyrimidin-4-one; and
3-[2-(3-fluoro-phenyl)-ethyl]-2-(2-hydroxy-phenyl)-3,5,6,7-tetrahydro-cyclopentapyrimidin-4-one.

As disclosed in International Patent Application Serial No. PCT/US2003/035162 titled Quinazolinone Compounds as Calcilytics which was filed on behalf of Shcherbakova et al., 3H-quinazolin-4-ones, which can be viewed as a structural combination of a 3H-pyrimidin-4-one and the annelated aromatic ring at 5 and 6 positions of the heterocyclic fragment, are active both in vitro and in vivo as calcilytics. Polysubstituted 3H-pyrimidin-4-ones with substituents at positions 5 and 6 other than the annelated aromatic ring are the structural derivatives of 3H-quinazolin-4-ones.

### EXAMPLES

The following specific examples are included for illustrative purposes only and are not to be considered as limiting to this disclosure. The reagents and intermediates used in the following examples are either commercially available or can be prepared according to standard literature procedures by those skilled in the art of organic synthesis.

Microwave reactions were performed on Emrys™ Optimizer (Biotage, Charlottesville, Va.) on continuous irradiation at 2450 MHz. All microwave reactions were carried out in heavy-walled Pyrex tubes, inner diameter 9 mm and height 147 mm, sealed with screw cap fitted Teflon Septa.

HPLC (High Pressure Liquid Chromatography) analyses for 98+% purity confirmation were performed on a Shimadzu RID-10A Series HPLC equipped with a SPD-M10A VP diode array detector, two LC-AT pumps, and a SIL-10A autoinjector using either an Altima C18 (5μ, 4.6×259 mm) or an Intersil ODS2 (5μ, 4.6×259 mm) column.

NMR (Nuclear Magnetic Resonance) spectroscopy was performed on a Varian Gemini 300 spectrometer. Proton and carbon spectra were recorded at 300 MHz and 75 MHz, respectively, in deuterochloroform (CDCl₃), methanol-d₄ (CH₃OH-d₄), or dimethylsulfoxide-d₆ (DMSO-d₆) solutions. NMR resonances are reported in δ (ppm) relative to tetramethylsilane (TMS) as internal standard with the following descriptors for the observed multiplicities: s (singlet), d (doublet), t (triplet), q (quartet), dd (doublet of doublets), and m (multiplet). J_{AB} coupling constants are reported in Hz.

Note that Examples 1-17 correspond with Examples 4-20 as presented in U.S. Provisional Application Ser. No. 60/479,323 which was filed on Jun. 18, 2003 and is titled Pyrimidinone Compounds as Calcilytics. The examples also correspond with those as presented in U.S. Patent Application Ser. No. 60/460,859 which was filed on Apr. 7, 2003 and is titled Pyrimidinone Compounds as Calcilytics. In particular, Examples 1-10 correspond with Examples 4-13 in Ser. No. 60/460,859 and Examples 12-14 correspond with Examples 14-16 in Ser. No. 60/460,859. Examples 1-17 also correspond with Examples 4-20 as presented in International Patent Application Ser. No. ______ which is titled Pyrimidinone Compounds as Calcilytics and which was filed on Apr. 7, 2004. These applications are all incorporated herein by specific reference.

### Example 1

### Preparation of 2-(2-Hydroxy-phenyl)-5,6-dimethyl-3-phenethyl-3H-pyrimidin-4-one

### a.) 2-(2-Methyl-[1,3]dioxolan-2-yl)-propionic acid ethyl ester

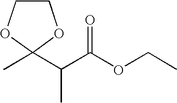

A mixture of 2-methyl-3-oxo-butyric acid ethyl ester (50 g, 0.347 mol), ethylene glycol (65 g, 1.05 mol) and p-toluenesulfonic acid monohydrate (0.2 g) in anhydrous toluene (200 mL) was refluxed using a Dean-Stark trap until the theoretical amount of water (6.3 mL) was collected. After cooling, the mixture was extracted with saturated bicarbonate solution (100 mL), water (100 mL×5), and brine (100 mL×2). After drying with sodium sulfate, filtration, and concentration on a rotary evaporator, the product was purified by distillation (fraction with b.p. 74-76° C./2 mm Hg) to yield 47.12 g (72%) of 2-(2-methyl-[1,3]dioxolan-2-yl)propionic acid ethyl ester.

¹H NMR (CDCl₃): δ 4.16 (q, 2H, J=7.2), 3.97 (m, 4H), 2.76 (q, 1H, J=7.2), 1.41 (s, 3H), 1.27 (t, 3H, J=7.2), 1.23 (d, 3H, J=7.2). ¹³C NMR (CDCl₃): δ 173.24, 109.76, 64.81, 60.38, 47.88, 21.29, 14.14, 12.83.

### b.) 2-(2-Methyl-[1,3]dioxolan-2-yl)-propionic acid

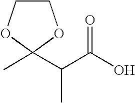

2-(2-Methyl-[1,3]dioxolan-2-yl)-propionic acid ethyl ester of Example 1a (31 g, 0.1647 mol) was dissolved in the mixture of dioxane—water (1:1, 350 mL) containing potassium hydroxide (35.63 g, 0.63 mol). The reaction mixture was stirred overnight at 35° C. and concentrated under high vacuum to give a white solid which was dissolved in water (200 mL) and extracted with dichloromethane (100 mL×2). The aqueous portion was acidified to pH=2 with 2N aqueous hydrochloric acid and the product was extracted with chloroform. The organic layer was washed with brine (300 mL), dried with sodium sulfate and concentrated under vacuum. 2-(2-methyl-[1,3]dioxolan-2-yl)-propionic acid was isolated as clear oil (20.34 g, 77%) which did not require further purification.

¹H NMR (CDCl₃): δ 11.29 (s, 1H), 4.01 (m, 4H), 2.80 (q, 1H, J=7.2), 1.43 (s, 3H), 1.26 (d, 3H, J=7.2). ¹³C NMR (CDCl₃): δ 178.61, 109.57, 64.84, 64.81, 47.80, 21.15, 12.61.

### c.) 2-(2-Methyl-[1,3]dioxolan-2-yl)-N-phenethyl-propionamide

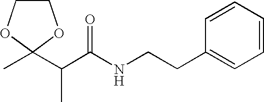

2-(2-Methyl-[1,3]dioxolan-2-yl)-propionic acid of Example 1b (1.60 g, 10 mmol) in dry dichloromethane (15 mL) was cooled to 0° C. under an argon atmosphere. A solution of oxalyl dichloride (2.92 g, 2.0 mL, 23.0 mmol) in dichloromethane (5 mL) was added dropwise. After 5 min at 0° C., the mixture was allowed to warm to room temperature. After stirring for 2 h at room temperature, the excess oxalyl dichloride was removed under reduced pressure to produce a yellow oil which was dissolved in dichloromethane (7 mL). The solution was cooled in an ice bath and phenethylamine (1.12 g, 10.0 mmol) in pyridine (5 mL) was added drop-wise. After the addition was complete, the reaction was warmed to room temperature and allowed to stir overnight. The solution was diluted with dicholoromethane (100 mL) and poured into ice-cold hydrochloric acid (1N, 150 mL). The organic layer was separated and washed with water (100 mL), sodium bicarbonate solution (5%, 50 mL), water (100 mL), and brine (100 mL). After drying with sodium sulfate and concentration on a rotary evaporator, the product was purified by flash chromatography on silica gel, eluting with hexanes—ethyl acetate (3:2) to give 2-(2-methyl-[1,3]dioxolan-2-yl)-N-phenethyl-propionamide (1.81 g, 69%) as colorless crystals.

¹H NMR (CDCl₃): δ 7.26 (m, 5H), 6.42 (broad s, 1H), 3.93 (m, 2H), 3.86 (m, 2H), 3.51 (m, 2H), 2.82 (t, 2H, J=7.2), 2.55 (q, 1H, J=7.2), 1.26 (s, 3H), 1.17 (d, 3H, J=7.2). ¹³C NMR (CDCl₃): δ 172.61, 138.99, 128.74, 128.43, 126.32, 109.77, 64.67, 64.50, 49.36, 40.45, 35.58, 21.20, 12.42.

### d.) 2-Methyl-3-oxo-N-phenethyl-butyramide

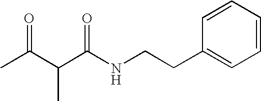

2-(2-Methyl-[1,3]dioxolan-2-yl)-N-phenethyl-propionamide of Example 1c (0.40 g, 1.5 mmol) was added to p-toluenesulfonic acid monohydrate (0.48 g, 2.5 mmol) in water (20 mmol) under a nitrogen atmosphere at room temperature. Acetone (20 mL) was added, and the reaction mixture was stirred overnight at room temperature and then heated at 95° C. for 3 h. After cooling to room temperature, the solution was made basic with sodium carbonate (0.5 g). The acetone was removed at room temperature under vacuum and the remaining aqueous material was extracted with dichloromethane (50 mL). The organic layer was washed with water (50 mL), brine (50 mL) and then dried with sodium sulfate. After concentration, the product was purified by crystallization from hexanes—ethyl acetate (1:1) to give 2-methyl-3-oxo-N-phenethyl-butyramide (0.17 g, 52%) as a white solid.

¹H NMR (CDCl₃): δ 7.26 (m, 5H), 6.13 (broad s, 1H), 3.52 (m, 2H), 3.34 (q, 1H, J=7.2), 2.81 (t, 2H, J=7.2), 2.19 (s, 3H), 1.34 (d, 3H, J=7.2). ¹³C NMR (CDCl₃): δ 207.27, 169.17, 138.54, 128.69, 128.61, 126.56, 54.98, 40.72, 35.55, 28.49, 14.58.

### e.) 3-Amino-2-methyl-but-2-enoic acid phenethyl-amide

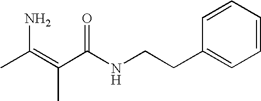

A solution of 2-methyl-3-oxo-N-phenethyl-butyramide of Example 1d (1.10 g, 5.00 mmol) in diethyl ether (300 mL) was saturated with gaseous ammonia for 3 h while cooled in an ice bath. Anhydrous aluminum chloride (99.99% purity, 0.667 g, 5.00 mmol) was then added in small portions and the reaction was allowed to stir at room temperature overnight. The reaction mixture was filtered and concentrated under reduced pressure to give 3-amino-2-methyl-but-2-enoic acid phenethyl-amide (0.97 g, 85% conversion by NMR) as a white solid which was used without purification for the next synthetic step.

### f.) Acetic acid 2-(1-methyl-2-phenethylcarbamoyl-propenylcarbamoyl)-phenyl ester

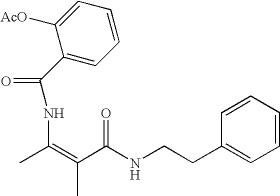

3-Amino-2-methyl-but-2-enoic acid phenethyl-amide of Example 1e (0.97 g, 5 mmol) was dissolved in tetrahydrofuran (20 mL) and pyridine (1.0 mL). Acetic acid 2-chlorocarbonyl-phenyl ester (0.993 g, 5.00 mmol) was added and the mixture was refluxed for 4 h. After cooling to room temperature, diethyl ether (50 mL) was added and the salts were removed by filtration. The filtrate was concentrated under reduced pressure. Additional ether (200 mL) was added and the remaining pyridine was extracted with 2N hydrochloric acid (3×30 mL). The ether was washed with brine (200 mL) and dried over anhydrous sodium sulfate. After concentration, the product was purified twice by flash chromatography on silica gel (118 g) eluting with hexanes—ethyl acetate (2:1) to give acetic acid 2-(1-methyl-2-phenethylcarbamoyl-propenylcaramoyl)-phenyl ester (0.58 g, 30%) as a yellow oil.

¹H NMR (CDCl₃): δ 12.95 (broad s, 1H), 7.82 (dd, 1H, J=7.8, 1.8), 7.48 (dt, 1H, J=7.5, 1.8), 7.31-7.12 (m, 7H), 5.71 (t, 1H, J=6.6), 3.54 (q, 2H, J=6.6), 2.82 (t, 2H, J=6.6), 2.44 (s, 3H), 2.31 (s, 3H), 1.74 (s, 3H). ¹³C NMR (CDCl₃): δ 169.83, 169.47, 164.15, 154.31, 148.80, 146.12, 138.71, 132.00, 129.50, 128.69, 126.58, 126.22, 123.50, 105.73, 40.56, 35.49, 21.06, 17.57, 13.25.

### g.) 2-(2-Hydroxy-phenyl)-5,6-dimethyl-3-phenethyl-3H-pyrimidin-4-one

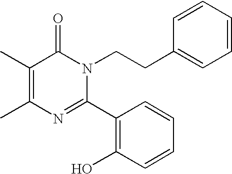

Acetic acid 2-(1-methyl-2-phenethylcarbamoyl-propenylcarbamoyl)-phenyl ester of Example 1f (220 mg, 0.59 mmol) was dissolved in the mixture of ethanol (8 mL) and water (8 mL) which contained 85% potassium hydroxide (0.80 g, 1.2 mmol). The mixture was refluxed overnight. After cooling, the reaction mixture was acidified with hydrochloric acid to pH=1 and extracted with dichloromethane (3×30 mL). The combined organic layers were washed with water (50 mL), brine (50 mL) and dried over anhydrous sodium sulfate. After concentration, the product was purified by flash chromatography on silica gel (39 g) eluting with hexanes—ethyl acetate (1:1) to give 2-(2-hydroxy-phenyl)-5,6-dimethyl-3-phenethyl-3H-pyrimidin-4-one (70 mg, 37%) as a white solid.

¹H NMR (CDCl₃): δ 7.15-7.10 (m, 4H), 7.01 (d, 1H, J=7.8), 6.82-6.78 (m, 3H), 6.71 (d, 1H, J=7.8), 4.07 (t, 1H, J=7.8), 2.80 (t, 2H, J=7.8), 2.22 (s, 3H), 2.08 (s, 3H). ¹³C NMR (CDCl₃): δ 162.60, 157.02, 155.81, 154.24, 137.59, 131.70, 129.06, 128.64, 128.42, 126.47, 120.82, 119.77, 119.22, 117.44, 47.77, 34.26, 20.89, 11.69.

### Example 2

### Preparation of 3-[2-(2-Fluoro-phenyl)-ethyl]-2-(2-hydroxy-phenyl)-5,6-dimethyl-3H-pyrimidin-4-one

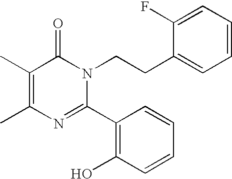

Utilizing the procedures described in Example 1-g except substituting 2-fluoro-phenethylamine for phenethylamine in step 1c the title compound was prepared as a white solid after crystallization from hexanes—ethyl acetate (3:1).

¹H NMR (CDCl₃): δ 9.88 (broad s, 1H), 7.11 (m, 2H), 6.91-671 (m, 6H), 4.09 (t, 2H, J=7.5), 2.88 (t, 2H, J=7.5), 2.23 (s, 3H), 2.08 (s, 3H). ¹³C NMR (CDCl₃): δ 162.69, 161.07 (d, J=243), 157.22, 155.90, 154.23, 131.53, 131.02 (d, J=4.2), 129.07, 128.34 (d, J=8.0), 124.52 (d, J=16), 124.00 (d, J=3.2), 120.77, 119.88, 119.66, 119.08, 116.99, 115.19 (d, J=22), 46.20, 27.51, 20.88, 11.61.

### Example 3

### Preparation of 3-[2-(3-Fluoro-phenyl)-ethyl]-2-(2-hydroxy-phenyl)-5,6-dimethyl-3H-pyrimidin-4-one

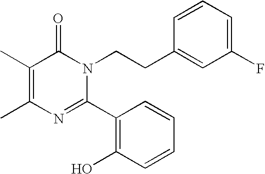

Utilizing the procedures described in Example 1a-g except substituting 3-fluoro-phenethylamine for phenethylamine in step Ic the title compound was prepared as a white solid after crystallization from hexanes—ethyl acetate (3:1).

¹H NMR (CDCl₃): δ 9.76 (broad s, 1H), 7.23 (m, 1H), 6.85 (m, 3H), 6.65 (d, 1H, J=7.8), 6.53 (m, 1H), 4.20 (t, 2H, J=7.5), 2.86 (t, 2H, J=7.5), 2.24 (s, 3H), 2.11 (s, 3H). ¹³C NMR (CDCl₃): δ 162.73 (d, J=244), 162.63, 156.65, 155.68, 154.64, 139.98 (d, J=7.5), 132.04, 129.94 (d, J=7.4), 128.90, 124.25, 120.15, 119.88, 119.29, 118.17, 115.51 (d, J=21), 113.52 (d, J=21), 47.57, 33.99, 20.95, 11.70.

### Example 4

### Preparation of 3-[2-(4-Fluoro-phenyl)ethyl]-2-(2-hydroxy-phenyl)-5.6-dimethyl-3H-pyrimidin-4-one

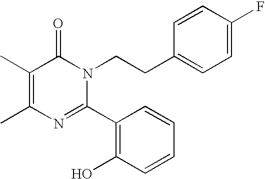

Utilizing the procedures described in Example 1a-g except substituting 4-fluoro-phenethylamine for phenethylamine in step 1c the title compound was prepared as a white solid after crystallization from hexanes—ethyl acetate (3:1).

¹H NMR (CDCl₃): δ 9.85 (broad s, 1H), 7.20 (m, 1H), 7.09 (dd, 1H, J₁=7.8, J₂=1.5), 6.81 (m, 6H), 4.13 (t, 2H, J=7.8), 2.80 (t, 2H, J=7.8), 2.23 (s, 3H), 2.10 (s, 3H). ¹³C NMR (CDCl₃): δ 162.66, 161.66 (d, J=243), 156.69, 155.76, 154.65, 133.24 (d, J=3.1), 132.02, 130.09 (d, J=8.3), 128.98, 120.38, 119,92, 119.32, 118.24, 115.33 (d, J=21), 47.86, 33.50, 20.96, 11.71.

### Example 5

### Preparation of 5-Ethyl-2-(2-hydroxy-phenyl)-6-methyl-3-phenethyl-3H-pyrimidin-4-one

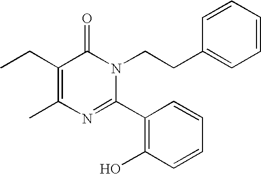

Utilizing the procedures described in Example 1a-g except substituting 2-ethyl-3-oxo-butyric acid ethyl ester for 2-methyl-3-oxo-butyric acid ethyl ester in step 1a the title compound was prepared.

¹H NMR (CDCl₃): δ 7.29-7.08 (m, 5H), 6.88-6.78 (m, 4H), 4.15 (t, 2H, J=7), 2.86 (t, 2H, J=7), 2.56 (q, 2H, J=7.5), 2.25 (s, 3H), 1.12 (t, 3H, J=7.5). ¹³C NMR (CDCl₃): δ 162.60, 156.52, 156.04, 155.20, 137.89, 132.08, 129.18, 128.82, 126.78, 125.10, 119.93, 118.05, 48.12, 34.58, 20.66, 19.87, 12.60.

### Example 6

### Preparation of 5-Ethyl-3-[2-(2-fluoro-phenyl)-ethyl]-2-(2-hydroxy-phenyl)-6-methyl-3H-pyrimidin-4-one

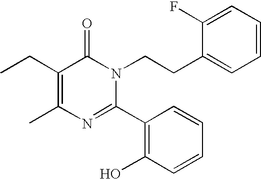

Utilizing the procedures described in Example 1a-g except substituting 2-ethyl-3-oxo-butyric acid ethyl ester for 2-methyl-3-oxo-butyric acid ethyl ester in step 1a and 2-fluoro-phenethylamine for phenethylamine in step 1c the title compound was prepared. Yield 51% after crystallization from hexanes—ethyl acetate (3:1).

¹H NMR (CDCl₃): δ 9.79 (broad s, 1H), 7.26-7.06 (m, 3H), 6.94-677 (m, 5H), 4.21 (t, 2H, J=7.2), 2.95 (t, 2H, J=7.2), 2.56 (q, 2H, J=7.6), 2.25 (s, 3H), 1.12 (t, 3H, J=7.6). ¹³C NMR (CDCl₃): δ 162.66, 161.38 (d, J=243), 156.33, 156.12, 155.372, 132.11, 131.29 (d, J=4.6), 129.13, 128.7 (d, J=7.8), 125.06, 124.71 (d, J=16.1), 124.33 (d, J=3.4), 119.91, 118.08, 115.51 (d, J=21), 46.78, 27.94, 20.64, 19.85, 12.59.

### Example 7

### Preparation of 5-Ethyl-3-[2-(3-fluoro-phenyl)-ethyl]-2-(2-hydroxy-phenyl)-6-methyl-3H-pyrimidin-4-one

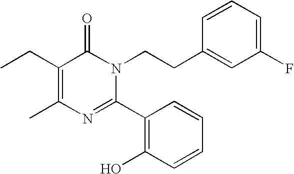

Utilizing the procedures described in Example 1a-g except substituting 2-ethyl-3oxo-butyric acid ethyl ester for 2-methyl-3-oxo-butyric acid ethyl ester in step 1a and 3-fluoro-phenethylamine for phenethylamine in step 1c the title compound was prepared. Yield 51% after crystallization from hexanes—ethyl acetate (3:1).

¹H NMR (300 CDCl₃): δ 9.66 (broad s, 1H), 7.26 (dt, 1H, J₁=8.0, J₂=1.5), 7.19-7.09 (m, 2H), 6.94-6.83 (m, 3H), 6.78 (d, 1H, J=7.7), 6.56 (dt, 1H, J₁=8.0, J₂=1.5), 4.23 (t, 2H, J=7.9), 2.89 (t, 2H, J=7.9), 2.57 (q, 2H, J=7.4), 2.27 (s, 3H), 1.14 (t, 3H, J=7.4). ¹³C NMR (CDCl₃): δ 162.83 (d, J=244), 162.36, 157.44, 156.06, 154.35, 140.33 (d, J=7.3), 131.73, 130.01 (d, J=7.9), 124.95, 124.53, 121.24, 119.73, 116.64, 115.67 (d, J=21), 113.53 (d, J=21), 47.42, 34.07, 20.47, 19.71, 12.44.

### Example 8

### Preparation of 5-Ethyl-3-[2-(4-fluoro-phenyl)-ethyl]-2-(2-hydroxy-phenyl)-6-methyl-3H-pyrimidin-4-one

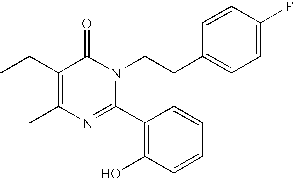

Utilizing the procedures described in Example 1a-g except substituting 2-ethyl-3-oxo-butyric acid ethyl ester for 2-methyl-3-oxo-butyric acid ethyl ester in step 1a and 4-fluoro-phenethylamine for phenethylamine in step 1c the title compound was prepared. Yield 51% after crystallization from hexanes-ethyl acetate (5:1).

¹H NMR (CDCl₃): δ 7.15 (dt, 1H, J₁=8.0, J₂=1.5), 7.06 (dd, 1H, J₁=7.8, J₂=1.5), 6.80-6.70 (m, 6H), 4.04 (t, 2H, J=7.5), 2.78 (t, 2H, J=7.5), 2.55 (q, 2H, J=7.5), 2.24 (s, 3H), 1.10 (t, 3H, J=7.5).

### Example 12

### Preparation of 2-(2-Hydroxy-phenyl)-5-methyl-3-phenethyl-6-trifluoromethyl-3H-pyrimidin-4-one

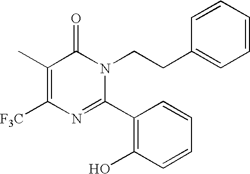

Utilizing the procedures described in Example 1a-g except substituting 2-trifluoromethyl-3-oxo-butyric acid ethyl ester for 2-methyl-3-oxo-butyric acid ethyl ester in step 1a the title compound was prepared. Yield 20% after three crystallizations from hexanes-ethyl acetate (2:1).

¹H NMR (CDCl₃): δ 10.31 (s, 1H), 7.42 (m, 1H), 7.19 (m, 3H), 7.13 (dd, 1H, J₁=7.6, J₂=1.6), 7.01 (d, 1H, J=7.9), 6.93 (m, 1H), 6.78 (m, 2H), 3.98 (t, 2H, J=7.8), 2.79 (t, 2H, J=7.8), 2.22 (q, 3H, J=2.2). ¹³C NMR (CDCl₃): δ 162.05, 156.90, 153.88, 144.91 (q, J=32), 137.61, 131.74, 129.66, 128.57, 128.33, 126.60, 122.40, 121.76 (q, J=275), 121.40, 119.22, 115.76, 47.50, 33.17, 10.78.

### Example 13

### Preparation of 2-(2-Hydroxy-phenyl)-3-phenethyl-5,6,7,8-tetrahydro-3H-quinazolin-4-one

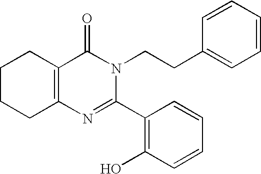

Utilizing the procedures described in Example 1a-g except substituting 2-oxo-cyclohexanecarboxylic acid ethyl ester for 2-methyl-3-oxo-butyric acid ethyl ester in step 1a the title compound was prepared. Yield 55% after crystallization from hexanes-ethyl acetate (1:1).

¹H NMR (CDCl₃): δ 10.00 (broad s, 1H), 7.14-7.00 (m, 5H), 6.80-6.69 (m, 4H), 4.02 (t, 2H, J=7.4), 2.79 (t, 2H, J=7.4), 2.5 (m, 4H), 1.68 (m, 4H).

¹³C NMR (CDCl₃): δ 162.42, 158.75, 156.29, 154.30, 137.87, 131.77, 129.36, 128.86, 128.59, 126.63, 121.33, 120.73, 119.85, 117.18, 47.60, 34.55, 30.79, 22.62, 21.97, 21.66.

Example 14

### Preparation of 3-[2-(3-Fluoro-phenyl)-ethyl]-2-(2-hydroxy-phenyl)-5,6,7,8-tetrahydro-3H-quinazolin-4-one

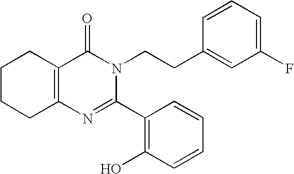

Utilizing the procedures described in Example 1a-g except substituting 2-oxo-cyclohexanecarboxylic acid ethyl ester for 2-methyl-3-oxo-butyric acid ethyl ester in step 1a and 3-fluoro-phenethylamine for phenethylamine in step 1c the title compound was prepared. Yield 56% after crystallization from hexanes-ethyl acetate (1:1).

¹H NMR (CDCl₃): δ 10.10 (broad s, 1H), 7.15-7.02 (m, 3H), 6.78-6.81 (m, 2H), 6.70 (d, 1H, J=8.1), 6.61 (d, 1H, J=7.7), 6.46 (d, 1H, J=8.1), 4.06 (t, 2H, J=7.0), 2.79 (t, 2H, J=7.0), 2.51 (m, 4H), 1.72 (m, 4H).

¹³C NMR (CDCl₃): δ 162.92 (d, J=244), 162.42, 158.63, 156.27, 154.38, 140.30 (d, J=7.3), 132.10, 130.14 (d, J=8.3), 129.34, 124.57 (d, J=2.2), 121.18, 120.85, 120.15, 118.02, 115.76 (d, J=20.7), 113.70 (d, J=21), 47.34, 34.25, 30.83, 22.68, 22.02, 21.71.

### Example 15

### Preparation of 5-Cyclopropyl-3-[2-(3-fluoro-phenyl)-ethyl]-2-(2-hydroxy-phenyl)-6-methyl-3H-pyrimidin-4-one

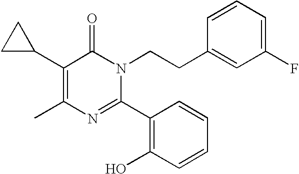

Utilizing the procedures described in Example 1a-g except substituting 2-cyclopropyl-3-oxo-butyric acid ethyl ester for 2-methyl-3-oxo-butyric acid ethyl ester in step 1a and 3-fluoro-phenethylamine for phenethylamine in step 1c the title compound was prepared. Yield 56% after crystallization from hexanes-ethyl acetate (1:1).

¹H NMR (CDCl₃): δ 9.70 (broad s, 1H), 7.31 (m, 1H), 7.15 (m, 2H), 6.91 (m, 3H), 6.70 (m, 1H), 6.59 (m, 1H), 4.25 (t, 2H, J=7.6), 2.90 (t, 2H, J=7.6), 2.38 (s, 3H), 1.61 (m, 1H), 0.99 (m, 2H), 0.87 (m, 2H). ¹³C NMR (CDCl₃): δ 162.77 (d, J=245), 162.35, 159.27, 156.16, 154.91, 140.05 (d, J=7.3), 132.10, 129.97 (d, J=8.1), 128.83, 124.34 (d, J=2.3), 122.95, 120.02, 119.82, 118.17, 115.55 (d, J=21), 113.56 (d, J=21), 47.40, 34.03, 21.22, 8.81, 6.64.

### Example 16

### Preparation of 2-(2-hydroxy-phenyl)-3-phenethyl-3,5,6,7-tetrahydro-cylopentapyrimidin-4-one

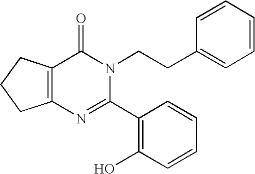

Utilizing the procedures described in Example 1a-g except substituting 2-oxo-cyclopentanecarboxylic acid ethyl ester for 2-methyl-3-oxo-butyric acid ethyl ester in step 1a the title compound was prepared. Yield 52% after crystallization from hexanes-ethyl acetate (1:1).

¹H NMR (CDCl₃): δ 9.12 (broad s, 1H), 7.17 (m, 5H), 6.85 (m, 4H), 4.18 (t, 2H, J=7.8), 2.84 (m, 6H), 2.08 (m, 2H). ¹³C NMR (CDCl₃): δ 166.59, 160.72, 158.96, 154.47, 137.61, 131.87, 128.98, 128.70, 128.51, 126.58, 123.61, 120.88, 119.86, 117.75, 47.58, 34.57, 34.33,27.83, 21.32.

### Example 17

### Preparation of 3-[2-(3-fluoro-phenyl)-ethyl]-2-(2-hydroxy-phenyl)-3,5,6,7-tetrahydro-cyclopentapyrimidin-4-one

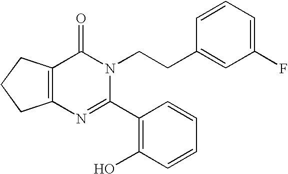

Utilizing the procedures described in Example 1a-g except substituting 2-oxo-cyclopentanecarboxylic acid ethyl ester for 2-methyl-3-oxo-butyric acid ethyl ester in step 1a and 3-fluoro-phenethylamine for phenethylamine in step 1c the title compound was prepared. Yield 51% after crystallization from hexanes-ethyl acetate (1:1).

¹H NMR (CDCl₃): δ 9.41 (broad s, 1H), 7.23 (m, 1H), 7.11 (m, 2H), 6.86 (m, 3H), 6.65 (d, 1H, J=7.6), 6.51 (d, 1H, J=9.6), 4.18 (t, 2H, J=7.7), 2.84 (m, 6H), 2.09 (m, 2H). ¹³C NMR (CDCl₃): δ 166.95, 162.74 (d, J=245), 160.64, 159.01, 154.20, 140.07 (d, J=7.4), 131.88, 129.96 (d, J=8.1), 128.99, 124.36, 123.61, 121.10, 119.86, 117.40, 115.56 (d, J=21), 113.53 (d, J=21), 113.53 (d, J=21), 47.14, 34.29, 34.19, 27.78, 21.29.

### Example 18

### Preparation of 3-[2-(3-Fluoro-phenyl)ethyl]-2-(2-hydroxy-phenyl)-5-isopropyl-6-methyl-3H-pyrimidin-4-one

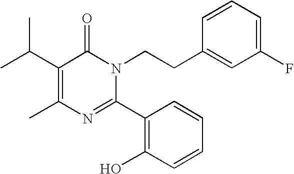

### a). 3-Amino-2-isopropyl-but-3-enoic acid methyl ester

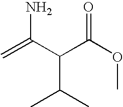

2-Methyl-3-oxo-butyric acid methyl ester (10 g, 0.0633 mol) was dissolved in absolute ethanol (50 mL). Excess of liquid ammonia (10 fold) was added and the mixture was stirred at room temperature in a sealed reaction vessel for 48 h. Excess ammonia and ethanol were removed under reduced pressure and the crude product (73% yield according to GC-MS data) was taken as such without further purification for the next synthetic step.

### b). 2-Isopropyl-3-(2-methoxy-benzoylamino)-but-3-enoic acid methyl ester

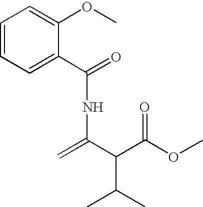

The crude 3-amino-2-isopropyl-but-3-enoic acid methyl ester of step 18a above in this method (5 g, 0.0318 mol) was dissolved in anhydrous THF (100 mL) and anhydrous pyridine (5.2 mL, 0.0637 mol) was added. Anisoyl chloride (4.28 mL, 0.0318 mol) was added dropwise, and the mixture was refluxed for 2 hours. After cooling, water (100 mL) was added and the organic layer was extracted with ethyl acetate (3×50 mL). The combined organic extracts were washed with 1N HCl (3×100 mL), water (100 mL), and brine (100 mL), dried over sodium sulfate and concentrated on a rotary evaporator. The product was purified by column chromatography over silica gel (200-400 mesh) eluting with 10% EtOAc/hexanes to give 2-isopropyl-3-(2-methoxy-benzoylamino)-but-3-enoic acid methyl ester (3 g, 33%) as a white powder.

¹H NMR (CDCl₃): δ 0.93 (d, 3H, J=6.6), 0.97 (d, 3H, J=6.6), 2.10-2.23 (m, 1H), 2.73 (d, 1H, J=11.1), 3.73 (s, 3H), 4.07 (s, 3H), 4.76 (d, 1H, J=1.2), 6.09 (s, 1H), 7.00 (d, 1H, J=8.1), 7.058-7.113 (m, 1H), 7.44-7.49 (m, 1H), 8.22 (dd, 1H, J=1.8, 6), 9.96 (br s, 1H). ¹³C NMR (CDCl₃): δ 19.9, 21.0, 29.3, 51.9, 55.8, 60.7, 103.8, 111.4, 121.3, 121.8, 132.4, 133.0,136.8, 157.4, 163.9, and 174.0.

### c). 3-[2-(3-Fluoro-phenyl)-ethyl]-5-isopropyl-2-(2-methoxy-phenyl)-6-methyl-3H-pyrimidin-4-one

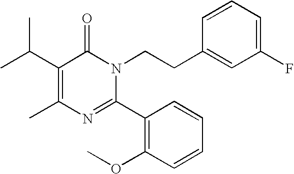

Phenyl magnesium bromide (1M solution in THF, 0.0021 mol) was added to a solution of 3-fluoro-phenethyl amine (0.27 mL, 0.0021 mol) in anhydrous toluene (20 mL). After stirring the mixture at 20° C. for 10 min, 2-isopropyl-3-(2-methoxy-benzoylamino)-but-3-enoic acid methyl ester of step 18b above in this method (0.05 g, 0.0017 mol) was added. (Note that 2-isopropyl-3-(2-methoxy-benzoylamino)-but-3-enoic acid methyl ester or more generrically 2-alkyl-3-(2-alkoxy-benzoylamino)-but-3-enoic acid methyl ester provides another example of an appropriate carbamide for forming 2,3,5,6-Substituted 3H-Pyrimidin-4-ones once cyclized). The mixture was refluxed for 10 hours, cooled and ethyl acetate (50 mL) was added followed by 1N HCl (50 mL). The organic layer was separated and the aqueous layer was extracted with EtOAc (3×50 mL). The combined organic extracts were washed with 1N HCl (3×100 mL), water (100 mL), and brine (100 mL). After drying over sodium sulfate and concentration on a rotary evaporator, the product was purified by column chromatography over silica gel (200-400 mesh) eluting with 12% EtOAc/hexanes to give 3-[2-(3-fluoro-phenyl)-ethyl]-5-isopropyl-2-(2-methoxy-phenyl)-6-methyl-3H-pyrimidin-4-one (0.3 g, 46%) as a white solid.

¹H NMR δ 1.30 (d, 1H, J=2.7), 1.31 (d, 1H, J=2.7), 2.28 (s, 3H), 2.64-2.82 (m, 2H), 3.01-3.16 (m, 1H), 3.45-3.55 (m, 1H), 3.71 (s, 3H), 4.16-4.25 (m, 1H), 6.40 (td, 1H, J=2.4, 9.6), 6.54 (d, 1H, J=7.8), 6.87-7.08 (m, 4H), 7.35-7.41 (m, 1H).

### d). 3-[2-(3-Fluoro-phenyl-ethyl]-2-(2-hydroxy-phenyl)-5-isopropyl-6-methyl-3H-pyrimidin-4-one

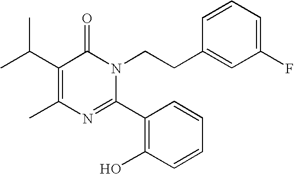

A dry heavy-walled Pyrex tube was charged with 3-[2-(3-fluoro-phenyl)-ethyl]-5-isopropyl-2-(2-methoxy-phenyl)-6-methyl-3H-pyrimidin-4-one of Example 18c (50 mg, 0.000132 mole), DMSO (5 mL) and sodium cyanide (65 mg, 10 equiv). The screw cap was tightened thoroughly. The reaction mixture was exposed to microwave irradiation at 180° C. for 1 hour. The reaction mixture was allowed to reach room temperature and was carefully acidified with 50% HCl and extracted with ethyl acetate (3×25 mL). Caution, HCN may form. The combined organic extracts were washed with water (50 mL), brine (50 mL), dried over anhydrous sodium sulfate, and concentrated. The crude product, which was almost pure, was filtered through a short column packed with silica gel (200-400 mesh) using 25% EtOAc/hexanes to afford 35 mg (72%) of 3-[2-(3-fluoro-phenyl)-ethyl]-2-(2-hydroxy-phenyl)-5-isopropyl-6-methyl-3H-pyrimidin-4-one. ¹H and ¹³C NMR spectral data of the compound were identical to those of the product prepared as described in Example 10.

It will be obvious to those having skill in the art that many changes may be made to the details of the above-described embodiments without departing from the underlying principles of the invention. The scope of the present invention should, therefore, be determined only by the following claims.

1. A method for preparing 2,3,5,6-Substituted 3H-Pyrimidin-4-ones comprising:
cyclizing an appropriate carbamide to obtain 2,3,5,6-Substituted 3H-Pyrimidin-4-ones.

2. The method as recited in claim 1, wherein the appropriate carbamide is an appropriate acetic acid 2-(1-alkyl-2-R⁴-carbamoyl-alk-1-enylcarbamoyl)-phenyl ester.

3. The method as recited in claim 2, further comprising: acylation of an appropriate 3-amino-2-alkyl-alk-2-enoic acid R⁴-amide to obtain the appropriate acetic acid 2-(1-alkyl-2-R⁴-carbamoyl-alk-1-enylcarbamoyl)-phenyl ester.

4. The method as recited in claim 3, further comprising: reacting 2-alkyl-3-oxo-R⁴-amide with anhydrous ammonia on catalysis by anhydrous aluminum chloride to obtain the appropriate 3-amino-2-alkyl-alk-2-enoic acid R⁴-amide.

5. The method as recited in claim 4, further comprising: reacting 2-(2-alkyl-[1,3]dioxolan-2-yl)-N-R⁴-alkanamide with p-toluenesulfonic acid monohydrate to obtain 2-alkyl-3-oxo-R⁴-amide.

6. The method as recited in claim 5, further comprising: reacting 2-(2-alkyl-[1,3]dioxolan-2-yl)-alkanoic acid with oxalyl chloride followed by reaction with a primary amine to obtain 2-(2-alkyl-[1,3]dioxolan-2-yl)-N-R⁴-alkanamide.

7. The method as recited in claim 6, further comprising: hydrolysis of 2-(2-alkyl-[1,3]dioxolan-2-yl)-alkanoic acid alkyl ester.

8. The method as recited in claim 7, further comprising: reacting of 2-alkyl-3-oxo-alkylic acid alkyl ester with ethylene glycol and p-toluenesulfonic acid monohydrate to obtain 2-(2-alkyl-[1,3]dioxolan-2-yl)alkanoic acid alkyl ester.

9. The method as recited in claim 1, wherein the appropriate carbamide is an appropriate 3-R³-carbamoylamino-2-alkyl-but-3-enoic acid methyl ester.

10. The method as recited in claim 9, wherein the 3-R³-carbamoylamino-2-alkyl-but-3-enoic acid methyl ester is cyclized by reacting it with a primary amine and the Grignard reagent.

11. The method as recited in claim 9, wherein the 3-R³-carbamoylamino-2-alkyl-but-3-enoic acid methyl ester is obtained by acylation of an appropriate 3-amino-2-alkyl-but-3-enoic acid methyl ester.

12. The method as recited in claim 11, wherein the appropriate 3-amino-2-alkyl-but-3-enoic acid methyl ester is obtained by reacting 2-alkyl-3-oxo-butyric acid methyl ester with liquid ammonia.

13. The method as recited in claim 1, wherein the appropriate carbamide is 2-alkyl-3-(2-alkoxy-benzoylamino)-but-3-enoic acid methyl ester.

14. The method as recited in claim 13, wherein the 2-alkyl-3-(2-alkoxy-benzoylamino)-but-3-enoic acid methyl ester is cyclized by reacting 2-alkyl-3-(2-alkoxy-benzoylamino)-but-3-enoic acid methyl ester with phenylmagnesium bromide and primary amine.

15. The method as recited in claim 13, wherein cyclizing the 2-alkyl-3-(2-alkoxy-benzoylamino)-but-3-enoic acid methyl ester yields 2-(2-alkoxy-phenyl)-3-R⁴-5,6-dialkyl-3H-pyrimidin-4-ones, and
wherein the method further comprises reacting 2-(2-alkoxy-phenyl)-3-R⁴-5,6-dialkyl-3H-pyrimidin-4-ones with sodium cyanide under microwave irradiation to yield 3-[2-(3-fluoro-phenyl)-ethyl]-2-(2-hydroxy-phenyl5-isopropyl-6-methyl-3H-pyrimidin-4-one.

16. The method as recited in claim 13, wherein the 2-alkyl-3-(2-alkoxy-benzoylamino)-but-3-enoic acid methyl ester is obtained by reacting 2-alkyl-3-oxo-butyric acid alkyl ester with liquid ammonia.

17. The method as recited in claim 1, wherein the 2,3,5,6-Substituted 3H-Pyrimidin-4-ones is at least one of:
2-(2-hydroxy-phenyl)-5,6-dimethyl-3-phenethyl-3H-pyrimidin-4-one;
3-[2-(2-fluoro-phenyl)-ethyl]-2-(2-hydroxy-phenyl5,6-dimethyl-3H-pyrimidin-4-one;
3-[2-(3-fluoro-phenyl)-ethyl]-2-(2-hydroxy-phenyl)-5,6-dimethyl-3H-pyrimidin-4-one;
3-[2-(4-fluoro-phenyl)-ethyl]-2-(2-hydroxy-phenyl)-5,6-dimethyl-3H-pyrimidin-4-one;
5-ethyl-3-[2-(2-fluoro-phenyl)-ethyl]-2-(2-hydroxy-phenyl)-6-methyl-3H-pyrimidin-4-one;
5-ethyl-3-[2-(3-fluoro-phenyl)-ethyl]-2-(2-hydroxy-phenyl)-6-methyl-3H-pyrimidin-4-one;
5-ethyl-3-[2-(4-fluoro-phenyl)-ethyl]-2-(2-hydroxy-phenyl)-6-methyl-3H-pyrimidin-4-one;
3-[2-(3-fluoro-phenyl)-ethyl]-2-(2-hydroxy-phenyl6-methyl-5-propyl-3H-pyrimidin-4-one;
3-[2-(3-fluoro-phenyl)-ethyl]-2-(2-hydroxy-phenyl5-isopropyl-6-methyl-3H-pyrimidin-4-one;
3-[2-(3-fluoro-phenyl)-ethyl]-2-(2-methoxy-phenyl)-5-isopropyl-6-methyl-3H-pyrimidin-4-one;
3-[2-(2-fluoro-phenyl)-ethyl]-2-(2-hydroxy-phenyl)-5-isopropyl-6-methyl-3H-pyrimidin-4-one;
2-(2-hydroxy-phenyl)-5-methyl-3-phenethyl-6-trifluoromethyl-3H-pyrimidin-4-one;
2-(2-hydroxy-phenyl)-3-phenethyl-5,6,7,8-tetrahydro-3H-quinazolin-4-one;
3-[2-(3-fluoro-phenyl)-ethyl]-2-(2-hydroxy-phenyl)-5,6,7,8-tetrahydro-3H-quinazolin-4-one;
5-cyclopropyl-3-[2-(3-fluoro-phenyl)-ethyl]-2-(2-hydroxy-phenyl)-6-methyl-3H-pyrimidin-4-one;
2-(2-hydroxy-phenyl)-3-phenethyl-3,5,6,7-tetrahydro-cyclopentapyrimidin-4-one; and
3-[2-(3-fluoro-phenyl)-ethyl]-2-(2-hydroxy-phenyl)-3,5,6,7-tetrahydro-cyclopentapyrimidin-4-one.

18. The method for preparing a compound having the chemical formula: 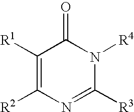
wherein:
R¹ and R² are either independently one of: H, halogen, CN, CF₃, lower alkyl, cycloalk, aryl; or R¹ and R² are together —(CH₂)ₙ- and n is 5, 4, or 3;
R³ is aryl group, which may have 1 to 4 substituents in the aryl ring each selected from the group consisting of: H, halogen, CN, CF₃, OCF₃, lower alkyl, N(lower alkyl)-₂, lower alkoxy, OH, OC(O)-lower alkyl, OC(O)-lower alkylamino, and OC(O)-lower alkyl-N(lower alkyl)₂;
R⁴ is H, lower alkyl, or a group of the formula —(CH₂)ₙ—R⁵ wherein n is 0, 1, or 2, and
R⁵ is an aryl group which may have 1 to 3 substituents on the aryl ring each selected from the group consisting of: H, halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, and OC(O)-lower alkyl-N(lower alk)₂;
and pharmaceutically acceptable salts or complexes; comprising: cyclizing an appropriate carbamide to yield the compound.

19. The method as recited in claim 18, wherein the appropriate carbamide is an appropriate acetic acid 2-(1-alkyl-2-R⁴-carbamoyl-alk-1-enylcarbamoyl)phenyl ester.

20. The method as recited in claim 19, further comprising: acylation of an appropriate 3-amino-2-alkyl-alk-2-enoic acid R⁴-amide to obtain the appropriate acetic acid 2-(1-alkyl-2-R⁴-carbamoyl-alk-1-enylcarbamoyl)-phenyl ester.

21. The method as recited in claim 20, further comprising: reacting 2-alkyl-3-oxo-R⁴-amide with anhydrous ammonia on catalysis by anhydrous aluminum chloride to obtain the appropriate 3-amino-2-alkyl-alk-2-enoic acid R⁴-amide.

22. The method as recited in claim 21, further comprising: reacting 2-(2-alkyl-[1,3]dioxolan-2-yl)-N-R⁴-alkanamide with p-toluenesulfonic acid monohydrate to obtain 2-alkyl-3-oxo-R⁴-amide.

23. The method as recited in claim 22, further comprising: reacting 2-(2-alkyl-[1,3]dioxolan-2-yl)-alkanoic acid with oxalyl chloride followed by reaction with a primary amine to obtain 2-(2-alkyl-[1,3]dioxolan-2-yl)-N-R⁴-alkanamide.

24. The method as recited in claim 23, further comprising: hydrolysis of 2-(2-alkyl-[1,3]dioxolan-2-yl)-alkanoic acid alkyl ester.

25. The method as recited in claim 24, further comprising: reacting of 2-alkyl-3-oxo-alkylic acid alkyl ester with ethylene glycol and p-toluenesulfonic acid monohydrate to obtain 2-(2-alkyl-[1,3]dioxolan-2-yl)-alkanoic acid alkyl ester.

26. The method as recited in claim 18, wherein the appropriate carbamide is an appropriate 3-R³-carbamoylamino-2-alkyl-but-3-enoic acid methyl ester.

27. The method as recited in claim 26, wherein the 3-R³-carbamoylamino-2-alkyl-but-3-enoic acid methyl ester is cyclized by reacting it with a primary amine and the Grignard reagent.

28. The method as recited in claim 26, wherein the 3-R³-carbamoylamino-2-alkyl-but-3-enoic acid methyl ester is obtained by acylation of an appropriate 3-amino-2-alkyl-but-3-enoic acid methyl ester.

29. The method as recited in claim 28, wherein the appropriate 3-amino-2-alkyl-but-3-enoic acid methyl ester is obtained by reacting 2-alkyl-3-oxo-butyric acid methyl ester with liquid ammonia.

30. The method as recited in claim 18, wherein the appropriate carbamide is 2-alkyl-3-(2-alkoxy-benzoylamino)-but-3-enoic acid methyl ester.

31. The method as recited in claim 30, wherein the 2-alkyl-3-(2-alkoxy-benzoylamino)-but-3-enoic acid methyl ester is cyclized by reacting 2-alkyl-3-(2-alkoxy-benzoylamino)-but-3-enoic acid methyl ester with phenylmagnesium bromide and primary amine.

32. The method as recited in claim 30, wherein cyclizing the 2-alkyl-3-(2-alkoxy-benzoylamino)-but-3-enoic acid methyl ester yields 2-(2-alkoxy-phenyl)-3-R⁴-5,6-dialkyl-3H-pyrimidin-4-ones, and
wherein the method further comprises reacting 2-(2-alkoxy-phenyl)-3-R⁴-5,6-dialkyl-3H-pyrimidin-4-ones with sodium cyanide under microwave irradiation to yield 3-[2-(3-fluoro-phenyl)-ethyl]-2-(2-hydroxy-phenyl)-5-isopropyl-6-methyl-3H-pyrimidin-4-one.

33. The method as recited in claim 30, wherein the 2-alkyl-3-(2-alkoxy-benzoylamino)-but-3-enoic acid methyl ester is obtained by reacting 2-alkyl-3-oxo-butyric acid alkyl ester with liquid ammonia.

34. The method as recited in claim 18, wherein the 2,3,5,6-Substituted 3H-Pyrimidin-4-ones is at least one of:
2-(2-hydroxy-phenyl)-5,6-dimethyl-3-phenethyl-3H-pyrimidin-4-one;
3-[2-(2-fluoro-phenyl)-ethyl]-2-(2-hydroxy-phenyl)-5,6-dimethyl-3H-pyrimidin-4-one;
3-[2-(3-fluoro-phenyl)-ethyl]-2-(2-hydroxy-phenyl)-5,6-dimethyl-3H-pyrimidin-4-one;
3-[2-(4-fluoro-phenyl)ethyl]-2-(2-hydroxy-phenyl)-5,6-dimethyl-3H-pyrimidin-4-one;
5-ethyl-2-(2-hydroxy-phenyl)-6-methyl-3-phenethyl-3H-pyrimidin-4-one;
5-ethyl-3-[2-(2-fluoro-phenyl)-ethyl]-2-(2-hydroxy-phenyl)-6-methyl-3H-pyrimidin-4-one;
5-ethyl-3-[2-(3-fluoro-phenyl)-ethyl]-2-(2-hydroxy-phenyl)-6-methyl-3H-pyrimidin-4-one;
5-ethyl-3-[2-(4-fluoro-phenyl)-ethyl]-2-(2-hydroxy-phenyl)-6-methyl-3H-pyrimidin-4-one;
3-[2-(3-fluoro-phenyl)-ethyl]-2-(2-hydroxy-phenyl)-6-methyl-5-propyl-3H-pyrimidin-4-one;
3-[2-(3-fluoro-phenyl)-ethyl]-2-(2-hydroxy-phenyl)-5-isopropyl-6-methyl-3H-pyrimidin-4-one;
3-[2-(3-fluoro-phenyl)-ethyl]-2-(2-methoxy-phenyl)-5-isopropyl-6-methyl-3H-pyrimidin-4-one;
3-[2-(2-fluoro-phenyl)-ethyl]-2-(2-hydroxy-phenyl)-5-isopropyl-6-methyl-3H-pyrimidin-4-one;
2-(2-hydroxy-phenyl)-5-methyl-3-phenethyl-6-trifluoromethyl-3H-pyrimidin-4-one;
2-(2-hydroxy-phenyl)-3-phenethyl-5,6,7,8-tetrahydro-3H-quinazolin-4-one;
3-[2-(3-fluoro-phenyl)-ethyl]-2-(2-hydroxy-phenyl)-5,6,7,8-tetrahydro-3H-quinazolin-4-one;
5-cyclopropyl-3-[2-(3-fluoro-phenyl)-ethyl]-2-(2-hydroxy-phenyl)-6-methyl-3H-pyrimidin-4-one;
2-(2-hydroxy-phenyl)-3-phenethyl-3,5,6,7-tetrahydro-cyclopentapyrimidin-4-one; and
3-[2-(3-fluoro-phenyl)-ethyl]-2-(2-hydroxy-phenyl)-3,5,6,7-tetrahydro-cyclopentapyrimidin-4-one.

35. The method as recited in claim 18, wherein R¹ and R² are each lower alkyl.

36. The method as recited in claim 35, wherein said lower alkyl is one of methyl, ethyl, propyl, cyclopropyl and isopropyl.

37. The method as recited in claim 35, wherein R² is methyl.

38. The method as recited in claim 18, wherein R¹ and R² together are —(CH₂)ₙ— and wherein n is 4 or 3.

39. The method as recited in claim 18, wherein R¹ and R² together are at least one of —(CH₂)₄— and —(CH₂)₃—.

40. The method as recited in claim 18, wherein R³ is phenyl optionally substituted with hydroxy.

41. The method as recited in claim 18, wherein
R⁴ further comprises the group —(CH₂)ₙ—R⁵; wherein n is 1 or 2; and
R⁵ is phenyl optionally substituted with 1 or 2 halogens.

42. The method as recited in claim 41, wherein n is 2 and said halogens are one of fluorine and chlorine.
